(19) United States
(12) Patent Application Publication
(10) Pub. No.: US 2026/0364224 A1
(43) Pub. Date: Nov. 19, 2026

(54) ULTRASONIC ATOMIZER

(71) Applicant: DONGGUAN YIH TEH ELECTRIC PRODUCTS CO., LTD., DongGuan City (CN)

(72) Inventor: Ming Jen Hsiao, Miaoli County (TW)

(21) Appl. No.: 19/674,184

(22) Filed: May 12, 2026

Related U.S. Application Data
(63) Continuation-in-part of application No. 18/937,160, filed on Nov. 5, 2024.
(63) Continuation-in-part of application No. 19/636,861, filed on Apr. 1, 2026.
Application No. 19/636,861 is a continuation-in-part of application No. 18/937,160, filed on Nov. 5, 2024.

(51) Int. Cl. A61L 9/14

(52) U.S. Cl. CPC A61L 9/14; A61L 2209/132

(57) ABSTRACT

An ultrasonic atomizer includes an atomizing module and an electrical plug electrically connected to the atomizing module. The atomizing module is configured to be detachably coupled to a liquid container. A piezoelectric vibration element of the atomizing module is configured to contact liquid supplied from a liquid-conducting element of the liquid container to atomize the liquid into mist. The electrical plug is rotatably coupled to the atomizing module to permit insertion into a corresponding electrical receptacle in different orientations while permitting the liquid container in an upright orientation during use.

### CROSS-REFERENCES TO RELATED APPLICATION

This application is a Continuation-in-Part of U.S. patent application Ser. No. 18/937,160, filed on Nov. 5, 2024; U.S. patent application Ser. No. 19/636,861, filed on Apr. 1, 2026; U.S. patent application Ser. No. 19/646,524, filed on Apr. 13, 2026; and U.S. patent application Ser. No. 19/651,528, filed on Apr. 17, 2026, the entire disclosures of which are incorporated herein by reference.

### BACKGROUND OF THE INVENTION

### 1. Field of the Invention:

The present invention relates to an atomizing device, and more particularly to an ultrasonic atomizer.

### 2. Description of the Related Art:

A conventional aroma diffuser uses an aromatic liquid container filled with aromatic liquid to emit fragrance molecules. A liquid-conducting element (wick) for absorbing liquid used in the aromatic liquid container is assembled for use in atomization by a piezoelectric vibration element of the aroma diffuser. The liquid-conducting element (wick) may become loosened during use and is not easy to contact the piezoelectric vibration element, and therefore the atomization effect is poor or not continuous.

Furthermore, due to difference in length, different materials, or possible deformation of the liquid-conducting elements of the different liquid containers, the liquid at the liquid-conducting elements, such as essential oil or aromatic liquid, cannot be reliably transmitted to the piezoelectric vibration element for atomization.

Conventional fragrance atomizers may include an electrical plug, such as a USB electrical plug or an AC electrical plug, configured to be inserted into a corresponding electrical receptacle to receive power. However, because the electrical plug is fixed in orientation relative to the fragrance atomizer, the fragrance atomizer may not be conveniently connectable to electrical receptacles arranged in various different orientations. As a result, difficulties may arise when attempting to connect the fragrance atomizer to electrical receptacles installed in different directions or positions.

### SUMMARY OF THE INVENTION

In view of the above problems of the conventional art, an ultrasonic atomizer is provided, the ultrasonic atomizer comprises an atomizing module and an electrical plug, wherein the atomizing module is electrically connected to the electrical plug and configured to be detachably coupled to a liquid container, the atomizing module including a piezoelectric vibration element configured to contact liquid supplied through a liquid-conducting element of the liquid container for atomization.

According to some embodiments of the present invention, the electrical plug is rotatably coupled to the atomizing module.

According to some embodiments of the present invention, a liquid container is detachably coupled to the atomizing module, and the electrical plug is rotatably coupled to the atomizing module such that the electrical plug is insertable into corresponding electrical receptacles arranged in different orientations while the liquid container is maintained in an upright orientation during use.

According to some embodiments of the ultrasonic atomizer of the present invention, the ultrasonic atomizer comprising an electrical plug, a control circuit, and an atomizing module;
the atomizing module comprising a piezoelectric vibration module, a mist-guiding tube, a hollow housing, a connecting tube;
the mist-guiding tube comprising a mist outlet at an upper portion thereof;
the hollow housing comprising a tubular neck extending inwardly from an upper portion thereof and an assembling seat at the lower portion thereof;
an upper end of the mist outlet of the mist-guiding tube being movably coupled to the tubular neck in the hollow housing; the piezoelectric vibration module comprising a piezoelectric vibration element having a plurality of micro-holes formed therein, the piezoelectric vibration module being coupled in the mist-guiding tube; an upper portion of the piezoelectric vibration module being connected to a lower end of the mist outlet; an upper portion of the connecting tube being coupled in a lower portion of the mist-guiding tube; an upper free end of the connecting tube being connected to a lower portion of the piezoelectric vibration module; the plurality of micro-holes of the piezoelectric vibration element, the mist outlet, the tubular neck, and the connecting tube collectively defining a communicating channel; a lower portion of the connecting tube being movably coupled to an upper portion of the assembling seat;
the electrical plug being coupled to a side of the hollow housing;
the electrical plug being electrically connected to the control circuit, and the control circuit being electrically connected to the piezoelectric vibration element of the piezoelectric vibration module via a conductive path.

Wherein the electrical plug is configured to be inserted into an electrical receptacle to provide power to the control circuit and the control circuit is configured to supplying power to the piezoelectric vibration element of the atomizing module via the conductive path.

In some embodiments, the control circuit comprises an oscillator circuit and a driver circuit and/or a step-down circuit.

In some embodiments, the electrical plug may including but not limited to, a USB plug or an AC power plug.

According to an embodiment of the ultrasonic atomizer of the present invention, wherein the atomizing module 51 is configured to be coupled to a liquid container, the liquid container is detachably coupled to the assembling seat, the liquid container may be, for example, an aromatic liquid container or an essential oil bottle or fragrance liquid bottle. The liquid container has a bottle opening and a liquid-conducting element. A first end portion of the liquid-conducting element is disposed in liquid within the liquid container, and a second end portion thereof extends outward from the bottle opening and passes through the connecting tube. A free end of the second end portion of the liquid-conducting element protrudes from the upper portion of the connecting tube so as to be connected to the lower portion of the piezoelectric vibration element. The liquid-conducting element has, for example, a porous structure or capillary action and is configured to transport liquid from within the liquid container to the free end of the second end portion of the liquid-conducting element exposed outside the bottle opening, so as to provide liquid, such as essential oil or aromatic liquid, for atomization by the piezoelectric vibration element. By virtue of one side of the mist-guiding tube at the mist outlet being movably coupled in the tubular neck in the hollow housing, the upper portion of the connecting tube being coupled in the lower portion of the mist-guiding tube, and the lower portion of the connecting tube being movably coupled to the upper portion of the assembling seat, the mist-guiding tube and the connecting tube are subjected to a downward gravitational force under the driving action of gravity, and at the same time the lower end of the mist outlet is driven to push downward against the piezoelectric vibration element of the piezoelectric vibration module to move downward and abut against the free end of the second end portion of the liquid-conducting element, thereby facilitating reliable transmission of liquid, so as to solve the problem that, due to differences in length, different materials, or possible deformation and loosening of the liquid-conducting elements of different liquid containers, the liquid at the free end of the liquid-conducting elements, such as essential oil or aromatic liquid, cannot be reliably transmitted to the piezoelectric vibration element for atomization. The ultrasonic atomizer of the present invention can continuously transmit the liquid at the free end of the second end portion of a liquid-conducting element of the liquid container to the piezoelectric vibration element for atomization.

According to embodiments of the ultrasonic atomizer of the present invention, wherein, the electrical plug is rotatably coupled to the atomizing module, which is configured to be coupled with a liquid container, such as an essential oil bottle or an aromatic liquid bottle. the ultrasonic atomizer of the present invention has the advantages that the electrical plug is configured to be inserted into a corresponding receptacle in various different orientations, so that the electrical plug is rotatable to maintain the liquid container in an upright orientation during use.

And in some embodiments, the piezoelectric vibration element of a piezoelectric vibration module is configured to abut against a free end of the second end portion of a liquid-conducting element of the liquid container and to contact liquid, the liquid may includes an essential oil or an aromatic liquid or other fragrance-containing liquid can be reliably transmitted to the piezoelectric vibration element for atomization.

### BRIEF DESCRIPTION OF THE DRAWINGS

FIG. 1 is a perspective view of an embodiment of the ultrasonic atomizer according to the present invention;

FIG. 2 is an exploded view of the atomizing module and the liquid container of FIG. 1;

FIG. 3 is another exploded view of the atomizing module and the liquid container of FIG. 1;

FIG. 4 is a cross-sectional view of FIG. 1;

FIG. 5 is a sectional view of the power plug AA according to FIG. 4.

FIG. 6 is a schematic view according to FIG. 5, showing that, when the power plug is rotated by 90 degrees, the protrusion of the protruding rail is movably engaged with the stoppers of the concave rail.

FIG. 7 is a schematic view according to FIG. 5, showing that, when the power plug is rotated by 180 degrees, the protrusion of the protruding rail is movably engaged with the stoppers of the concave rail.

FIG. 8 is a schematic view according to FIG. 5, showing that, when the power plug is rotated by 270 degrees, the protrusion of the protruding rail is movably engaged with the stoppers of the concave rail;

FIG. 9 is another perspective view of an embodiment of the ultrasonic atomizer according to the present invention;

FIG. 10 is another exploded view of the atomizing module and the liquid container of FIG. 9;

FIG. 11 is another exploded view of the atomizing module and the liquid container of FIG. 9; and

FIG. 12 is another cross-sectional view of FIG. 9.

### DETAILED DESCRIPTION OF THE PREFERRED EMBODIMENT

As used herein, terms such as “including,” “comprising,” and “having” are intended to be inclusive and mean that additional elements may also be present.

As used herein, directional terms such as “upper,” “lower,” “upward,” and “downward” are used for illustrative purposes only and are not intended to limit the scope of the invention.

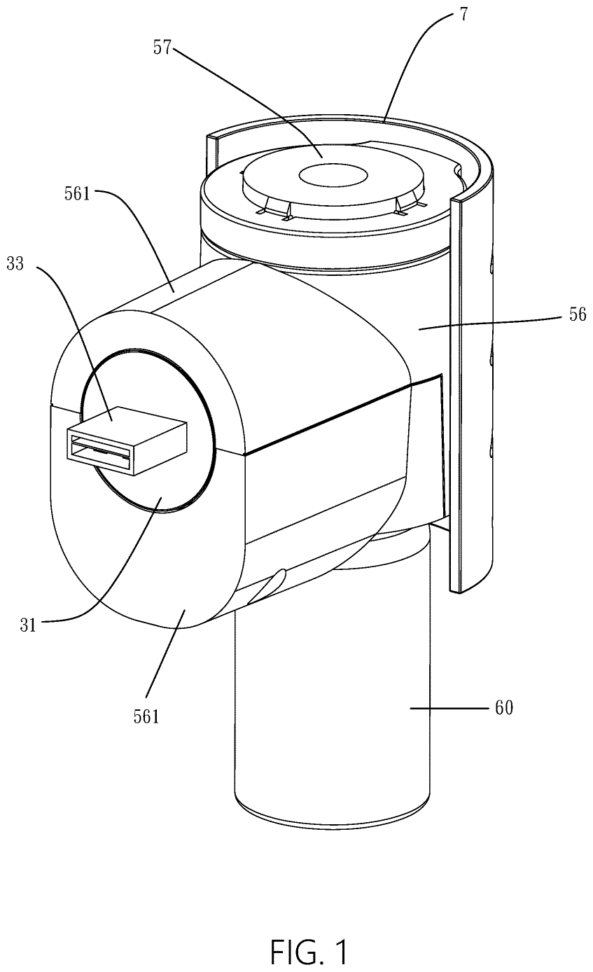

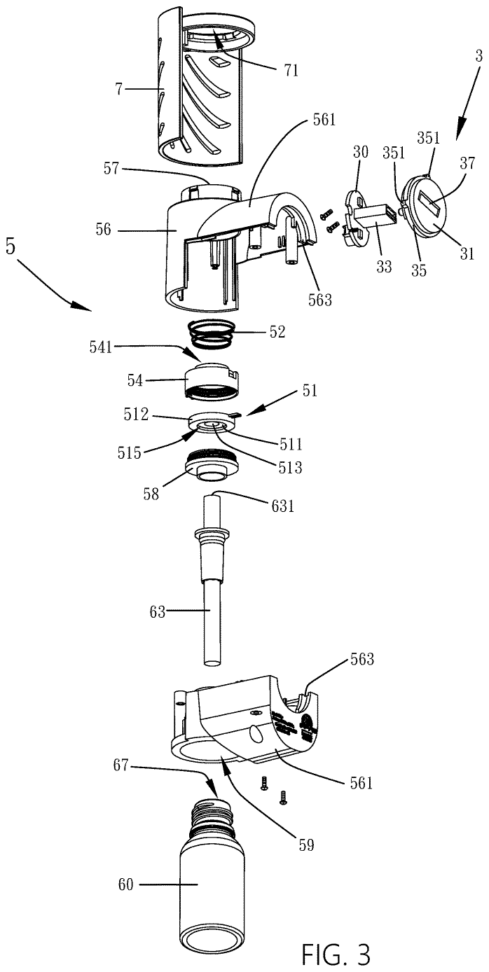

Please refer to FIGS. 1 to 4. In some embodiments of an ultrasonic atomizer of the present invention, the ultrasonic atomizer comprising an electrical plug 3, a control circuit 30, and an atomizing module 5;
the atomizing module 5 comprises a piezoelectric vibration module 51, a mist-guiding tube 54, a hollow housing 56, a connecting tube 58; the mist-guiding tube 54 comprising a mist outlet 541 at an upper portion thereof;
the hollow housing 56 comprising a tubular neck 57 extending inwardly from an upper portion thereof and an assembling seat 59 at the lower portion thereof;
an upper end of the mist outlet 541 of the mist-guiding tube 54 being movably coupled to the tubular neck 57 in the hollow housing 56; the piezoelectric vibration module 51 comprising a piezoelectric vibration element 511 having a plurality of micro-holes 513 formed therein, the piezoelectric vibration module 51 being coupled in the mist-guiding tube 54; an upper portion of the piezoelectric vibration module 51 being connected to a lower end of the mist outlet 541; an upper portion of the connecting tube 58 is coupled in a lower portion of the mist-guiding tube 54; an upper free end of the connecting tube 58 being connected to a lower portion of the piezoelectric vibration module 51; the plurality of micro-holes 513 of the piezoelectric vibration element 511, the mist outlet 541, the tubular neck 57, and the connecting tube 58 collectively defining a communicating channel;
a lower portion of the connecting tube 58 being movably coupled to an upper portion of the assembling seat 59;
the electrical plug 3 being coupled to a side of the hollow housing 56;
the electrical plug 3 being electrically connected to the control circuit 30, and the control circuit 30 being electrically connected to the piezoelectric vibration element 511 of the piezoelectric vibration module 51 via a conductive path.

Wherein the electrical plug 3 is configured to be inserted into an electrical receptacle to provide power to the control circuit 30 and the control circuit 30 is configured to supply power to the piezoelectric vibration element 511 of the atomizing module 5 via the conductive path.

In some embodiments of the ultrasonic atomizer of the present invention, the control circuit comprises an oscillator circuit and a driver circuit and/or a step-down circuit for driving the piezoelectric vibration element 511 of the atomizing module 5 to vibrate (not shown in drawings).

In some embodiments of the ultrasonic atomizer of the present invention, the electrical connection may be implemented by any suitable conductive structure, including but not limited to conductive wires, spring contacts, electrical contacts, electrodes, terminals or conductive traces.

Please refer to FIGS. 1 to 4. According to an embodiment of the ultrasonic atomizer of the present invention, wherein the atomizing module 51 is configured to be coupled to a liquid container 60. The liquid container 60 is detachably coupled to the assembling seat 59, for example by screwing, snap engagement, or other suitable coupling manner. The liquid container 60 for example, an aromatic liquid bottle or an essential oil bottle. The liquid container 60 has a bottle opening 67 and a liquid-conducting element 63. A first end portion of the liquid-conducting element 63 is disposed in liquid within the liquid container 60, and a second end portion thereof extends outward from the bottle opening 67 and passes through the connecting tube 58. A free end 631 of the liquid-conducting element 63 protrudes from an upper portion of the connecting tube 58 so as to be connected to the lower portion of the piezoelectric vibration element 511.

The liquid-conducting element 63 has, for example, a porous structure or capillary action and is configured to transport liquid from within the liquid container 60 to the free end 631 of the second end portion of the liquid-conducting element 63 exposed outside the bottle opening 67, so as to provide liquid, such as essential oil or aromatic liquid, for atomization by the piezoelectric vibration element 511. By virtue of the upper end of the mist-guiding tube 54 being movably sleeved on a lower portion of the tubular neck 57 in the hollow housing 56, the upper portion of the connecting tube 58 being coupled in the lower portion of the mist-guiding tube 54, and the lower portion of the connecting tube 58 being movably received in the upper portion of the assembling seat 59, the mist-guiding tube 54 and the connecting tube 58 are subjected to a downward gravitational force under the driving action of gravity, and at the same time the lower end of the mist outlet 541 is driven to push downward against the piezoelectric vibration element 511 of the piezoelectric vibration module 51 to move downward and abut against the free end 631 of the second end portion of the liquid-conducting element 63, thereby facilitating reliable transmission of liquid, so as to solve the problem that, due to differences in length, different materials, or possible deformation and loosening of the liquid conducting element 63 of different liquid containers, the liquid at the free end 631 thereof, such as essential oil or aromatic liquid, cannot be reliably transmitted to the piezoelectric vibration element 511 for atomization.

In some embodiments, the liquid container for example a liquid bottle or an essential oil bottle or an aromatic liquid bottle or fragrance liquid bottle.

In some embodiments of the ultrasonic atomizer of the present invention, the liquid-conducting element 63 comprises any one of a cotton wick, a PET fiber rod, a paper rod, a foam rod, or a porous liquid-conducting element made of a material having a porous structure or capillary action.

Please refer to FIGS. 1 to 4. In some embodiments of the ultrasonic atomizer of the present invention, the atomizing module 5 further comprises an elastic member 52 (for example, a compression spring), the elastic member 52 being compressed within the hollow housing and disposed between the upper portion of the hollow housing 56 and the upper portion of the mist-guiding tube 54 to urge the mist-guiding tube 54 downwardly. By virtue of one side of the mist-guiding tube 54 at the mist outlet 541 being movably sleeved on the lower portion of the tubular neck 57 in the hollow housing 56, the upper portion of the connecting tube 58 being coupled in the lower portion of the mist-guiding tube 54, and the lower portion of the connecting tube 58 being movably received to the upper portion of the assembling seat 59, the elastic force of the elastic member 52 is used to movably drive the mist-guiding tube 54 and the connecting tube 58 downward, while the lower end of the mist outlet 541 pushes downward against the piezoelectric vibration element 511 of the piezoelectric vibration module 51 so as to further move downward and more tightly abut against the free end 631 of the second end portion of the liquid-conducting element 63 protruding from the bottle opening of the liquid container 60, so that the liquid, such as essential oil or aromatic liquid, at the free end 631 of the second end portion of the liquid-conducting element 63 can be more tightly and effectively transmitted to the piezoelectric vibration element 511 for atomization.

In some embodiments, wherein a gap 501 is predetermined between the lower portion of the connecting tube 58 and the upper portion of the assembling seat 59. The tension of the elastic member 52 drives the mist-guiding tube 54 and the connecting tube 58 downward, while the lower end of the mist outlet 541 pushes downward against the piezoelectric vibration element 511 of the piezoelectric vibration module 51 so that the piezoelectric vibration element 511 moves downward within the distance of the gap 501, so as to movably adjust the downward abutting contact of the piezoelectric vibration element 511 against the free end 631 of the second end portion of the liquid-conducting element 63. By means of the above embodiment of the ultrasonic atomizer of the present invention, the piezoelectric vibration element 511 of the piezoelectric vibration module 51 is allowed to continuously and effectively contact, in a downward manner, the essential oil or liquid at the free end 631 of the second end portion of the liquid-conducting element 63, so as to maintain a proper liquid film thickness at the atomization interface between the free end of the second portion of the liquid-conducting element 63 and the piezoelectric vibration element 511 and continually atomize the liquid.

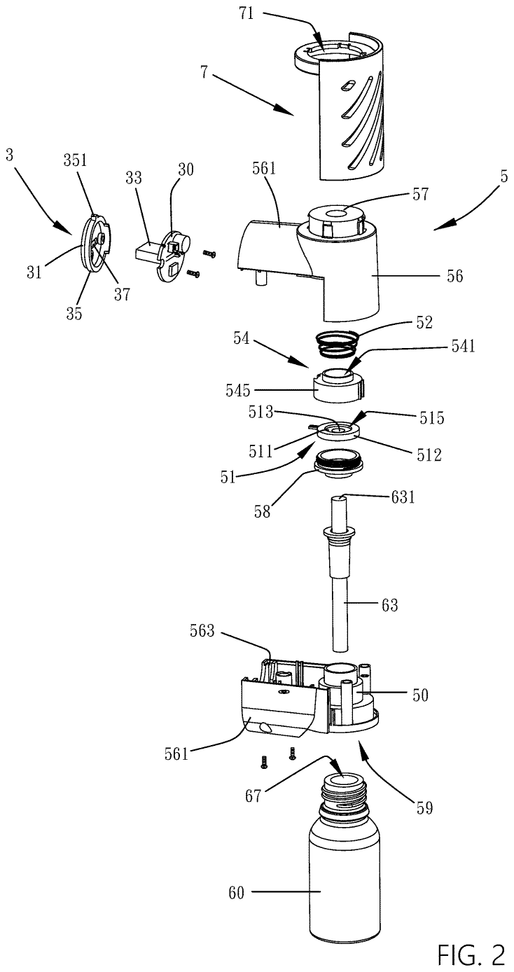

Please refer to FIGS. 2 to 4. In some embodiments of the ultrasonic atomizer of the present invention, the piezoelectric vibration module 51 comprises a fixing sleeve 512, and the fixing sleeve 512 is provided with an opening 515. The fixing sleeve 512 may be made of silicone, plastic, rubber, or any of various waterproof or insulating materials. In the illustrated embodiment, the piezoelectric vibration element 511 is, for example, a truncated-cone-shaped vibration sheet made of nickel alloy or iron alloy, and the plurality of micro-holes 513 are formed at a center of the truncated-cone-shaped vibration sheet. An annular piezoelectric ceramic plate may be coupled to the truncated cone-shaped vibration sheet, and an opening of the annular piezoelectric ceramic plate passes around an outer periphery of the plurality of micro-holes 513, so that the plurality of micro-holes 513 are located within the opening of the annular piezoelectric ceramic plate and are assembled as the piezoelectric vibration element 511. The piezoelectric vibration element 511 is disposed in the fixing sleeve 512. The plurality of micro-holes 513 of the piezoelectric vibration element 511 correspond to the opening 515 of the fixing sleeve 512. The upper portion of the opening 515 is connected to the lower end of the mist outlet 541, and the lower portion of the opening 515 is connected to the upper portion of the connecting tube 58.

In some embodiments, the diameter of the free end 631 of the second end portion of the liquid-conducting element 63 is smaller than the diameter of the opening 515 of the fixing sleeve 512, whereby the free end 631 of the second end portion of the liquid-conducting element 63 can be coupled in the opening 515 and contact the piezoelectric vibration element 511 and the plurality of micro-holes 513 thereof in a circumferentially confined manner, so as to more effectively deliver liquid to the piezoelectric vibration element 511.

Please refer to FIG. 2 to 4. In some embodiments of the ultrasonic atomizer of the present invention, A protruding rib 545 is formed on an outer side of the mist-guiding tube 54. The elastic member 52 comprises any one of a spring, a compression spring, an elastic block, or an elastic plate, and in the embodiments the elastic member 52 is a spring. One side of the spring storing elastic force is sleeved around the outer periphery of a lower portion of the tubular neck 57, and another side of the spring is sleeved on the upper portion of the mist-guiding tube 54 and abuts against the protruding rib 545, whereby the spring tension can drive the mist-guiding tube 54 and the connecting tube 58 downward, and simultaneously drive the lower end of the mist outlet 541 of the mist-guiding tube 54 to push the piezoelectric vibration element 511 of the piezoelectric vibration module 51 downward to tightly abut against the free end 631 of the liquid-conducting element 63.

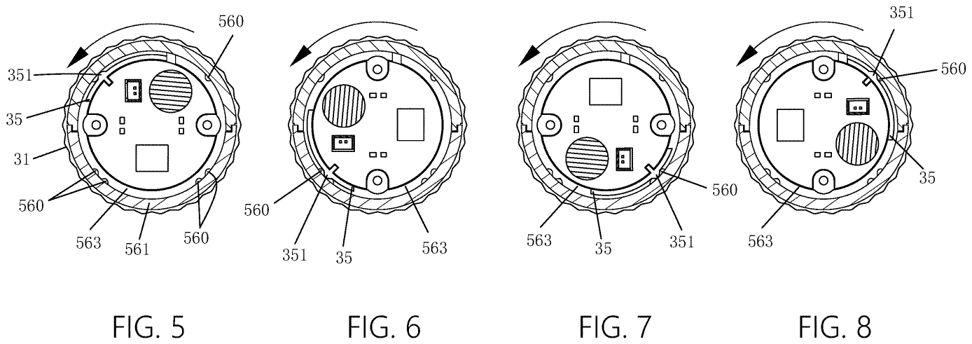

Please refer to FIGS. 1, 4 and 5-8. In some embodiments of the ultrasonic atomizer of the present invention, the electrical plug 3 is an angle-adjustable plug, the electrical plug 3 comprises a rotating portion 31 and a USB plug 33, the rotating portion 31 includes an annular rail 35 formed on an outer peripheral edge of the rotation portion 31, and the rotating portion is formed with at least a plug hole 37. The USB plug 33 may be, for example, a Type-A, Type-C, or Micro-USB plug. The USB plug 33 is coupled in the plug hole 37 to form the rotatable electrical plug 3. One side of the hollow housing 56 extends with a connecting pipe 561, an inner side of the connecting pipe 561 is formed with a concave rail 563, and the annular rail 35 of the rotating portion 31 is rotatably coupled in the concave rail 563 on the inner side of the connecting pipe 561. Thereby, when the ultrasonic atomizer of the present invention uses the USB plug 33 to be electrically connected in a USB electrical receptacle (not show in the drawings) of an unfavorable orientation, the Ultrasonic atomizer can rotate by an angle through the rotating portion 31 and the USB plug 33 relative to the USB electrical receptacle, so that the atomizer and the liquid bottle 60 can be maintained at an upward angle during use, thereby overcoming the problem that an USB plug of the atomizer is electrically connected for use with USB electrical receptacles arranged in different orientations. wherein the control circuit comprises, an oscillator circuit and a driver circuit for driving the piezoelectric vibration element 511 of the atomizing module 5 to vibrate (not shown in drawings).

In some embodiments, the rotatable electrical plug 3 is configured to plug into an electrical receptacle in various different orientations. In the embodiments, the electrical plug 3 is rotatably assembled to the atomizing module 5 so that its angle can be changed. The electrical plug 3 is configured to be detachably coupled to a USB electrical receptacle (not show in the drawings) in different orientations, such that the ultrasonic atomizer of the present invention can rotate by an angle relative to the USB electrical receptacle, for example, between about 1 degree to 360 degrees, whereby the electrical plug is rotatable to maintain the liquid container in an upright orientation during use.

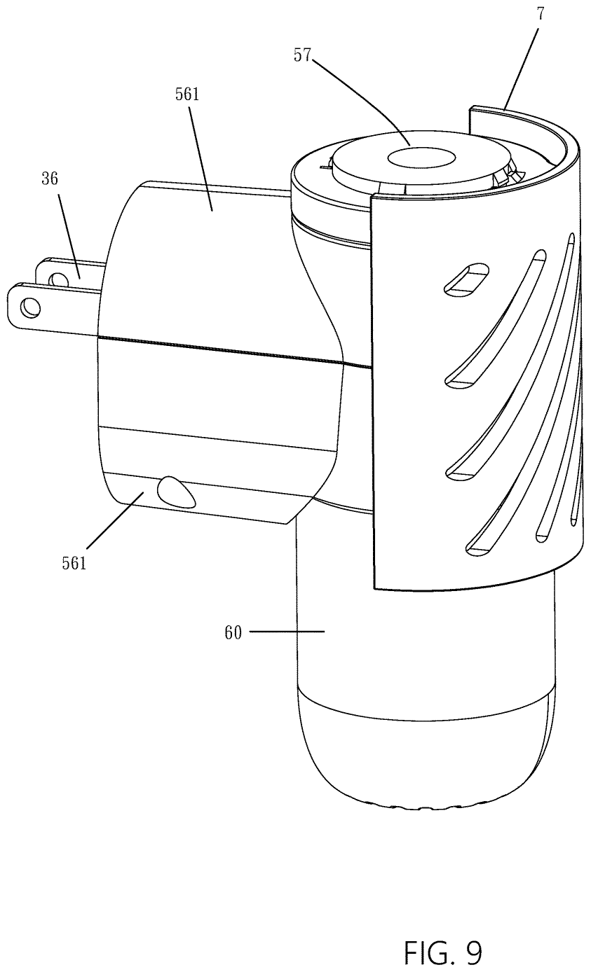

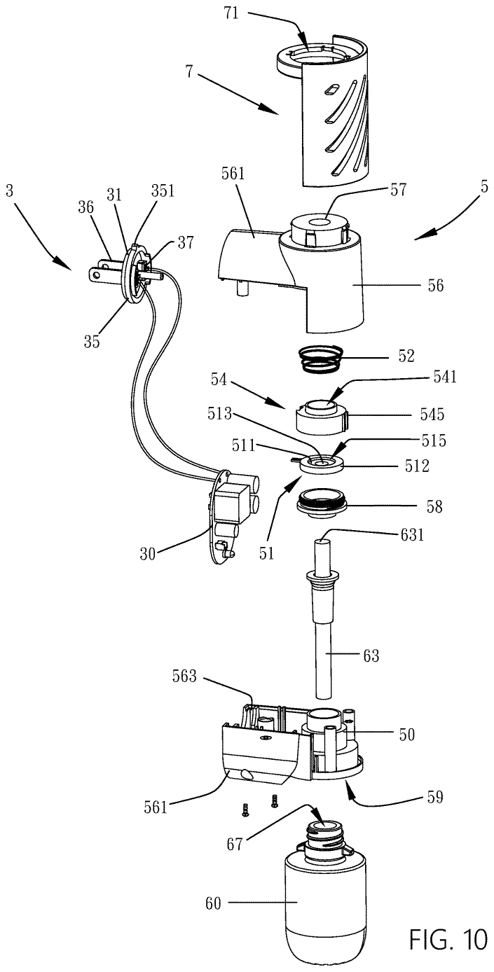

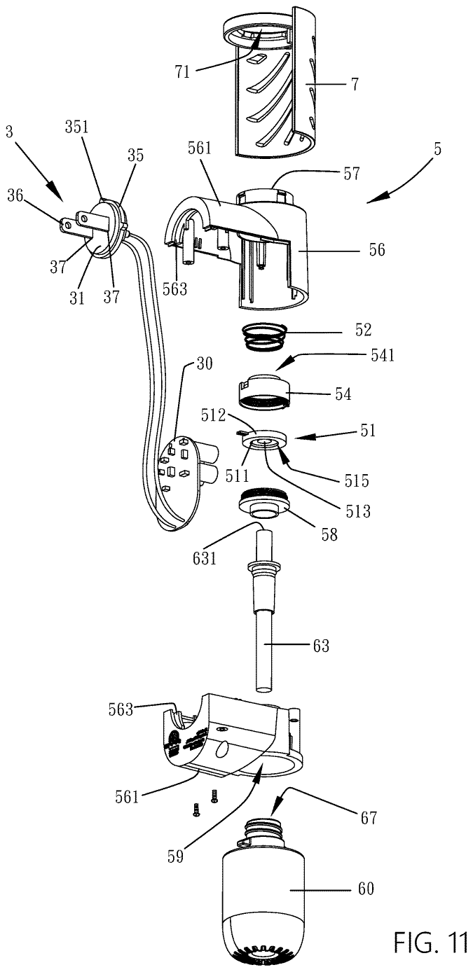

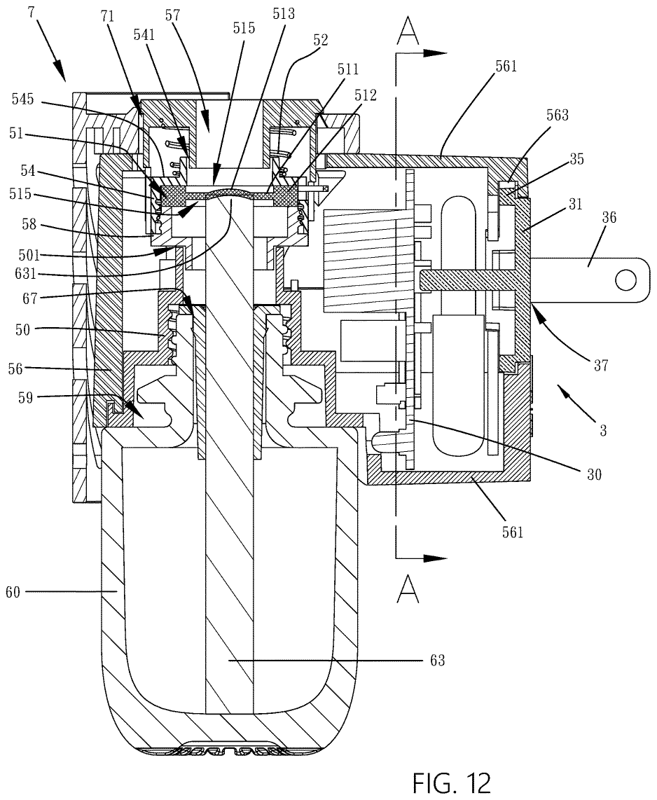

Please refer to FIGS. 9 to 12, In some embodiments of the ultrasonic atomizer of the present invention, wherein, the differences between the present embodiment and the previous embodiment include that the electrical plug 3 is configured as an AC plug 36 and that the control circuit further comprises a step-down circuit. Other structures, electrical connections, and operational features are substantially identical. The electrical plug 3 comprises a rotating portion 31 and an AC plug 36, the rotating portion 31 includes an annular rail 35 formed on an outer peripheral edge, and the rotating portion 31 is formed with two plug holes 37, the AC plug 36 being coupled in the plug holes 37; wherein one side of the hollow housing 56 extends with a connecting pipe 561, the inner side of the connecting pipe 561 is formed with a concave rail 563, and the annular rail 35 of the rotating portion 31 is rotatably coupled in the concave rail 563 on the inner side of the connecting pipe 561, wherein the control circuit 30 comprises, an oscillator circuit, a driver circuit and a step-down circuit for driving the piezoelectric vibration element 511 of the atomizing module 5 to vibrate (not shown in drawings).

In some embodiments, the rotatable electrical plug 3 is configured to plug into an electrical receptacle in various different orientations. In the embodiments, the electrical plug 3 is rotatably assembled to the atomizing module 5 so that its angle can be changed. The electrical plug 3 is configured to be detachably coupled to an Ac electrical receptacle (not show in the drawings) in different orientations, such that the ultrasonic atomizer of the present invention can rotate by an angle relative to the Ac electrical receptacle, for example, between about 1 degree to 360 degrees, whereby the electrical plug is rotatable to maintain the liquid container in an upright orientation during use.

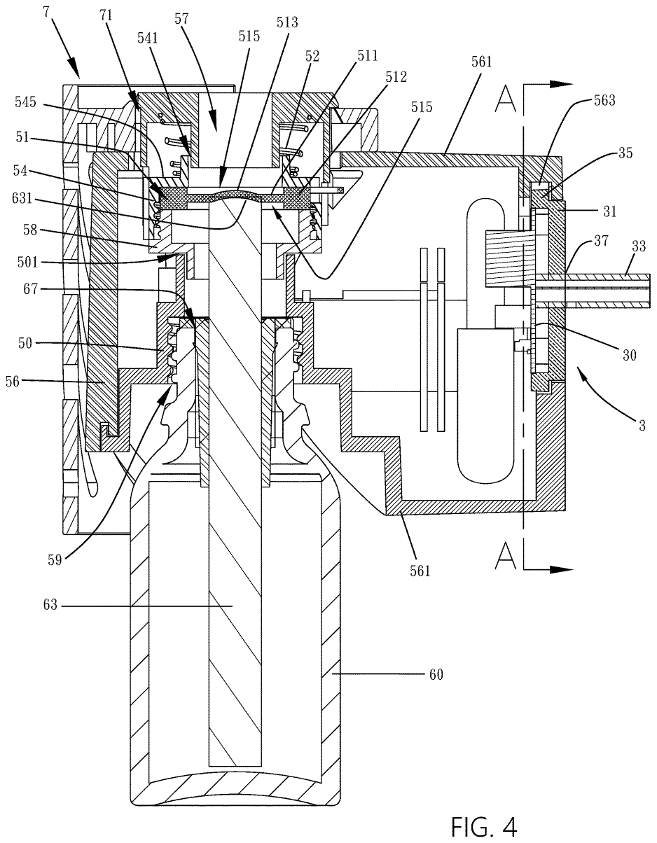

Please refer to FIGS. 4 to 8 and 12, In some embodiments of the ultrasonic atomizer of the present invention, the annular rail 35 of the rotating portion 31 may be configured to be tightly fitted in the concave rail 563 of the connecting pipe 561, wherein a damping or frictional effect generated when the annular rail 35 and the concave rail 563 relatively rotate is used to movably fix the connection angle between the electrical plug 3 and the hollow housing 56, for example including but not limited to 90 degrees or 180 degrees or 270 degrees.

In some embodiments, a protrusion 351 is formed on the surface of the annular rail 35, and correspondingly, at least a stopper 560 is provided at a 90 degrees or 180 degrees or 270 degrees position of the concave rail 563, whereby when the rotating portion 31 drives the annular rail 35 and the protrusion 351 thereof to rotate to one side in a 90 degrees or 180 degrees or 270 degrees direction, the protrusion 351 is movably engaged with the stopper 560 of the concave rail 563, please refer to FIG. 6 is a schematic view showing that, when the electrical plug 3 is rotated by 90 degrees, the protrusion 351 of the annular rail 35 is movably engaged with the stoppers 560 of concave rail 563, FIG. 7 is a schematic view showing that, when the electrical plug 3 is rotated by 180 degrees, the protrusion 351 of the annular rail 35 is movably engaged with the stoppers 560 of the concave rail 563, and FIG. 8 is a schematic view showing that, when the electrical plug 3 is rotated by 270 degrees, the protrusion 351 of the annular rail 35 is movably engaged with the stoppers 560 of concave rail 563 in some embodiments,

In some embodiments wherein two stoppers are formed at 90 degrees or 180 degrees or 270 degrees position of the concave rail, whereby when the rotating portion drives the annular rail and the protrusion thereof to rotate to one side in 90 degrees or 180 degrees or 270 degrees direction, the protrusion is movably engaged with the stoppers.

The angle-adjustable electrical plug 3 can rotate in 90 degrees or 180 degrees or 270 degrees direction relative to the mist outlet 541 and the liquid bottle 60 coupled with the hollow housing 56, whereby the electrical plug 3 after being rotated by a degree can be plugged into an electrical receptacle of a different orientation (not shown in drawings), thereby completing the ultrasonic atomizer of the present invention can rotate by a degrees relative to the electrical receptacle.

Please refer to FIGS. 1 to 4, and 9 to 12. In some embodiments of the ultrasonic atomizer of the present invention, wherein the atomizer further comprises a cover 7. The cover 7 is formed with a first opening 71 on the upper portion thereof. The cover 7 is coupled to the hollow housing 56, the first opening is sleeved around the out periphery of an upper portion of the tubular neck 57, while covering the atomizing module 5, The tubular neck 57 communicates with the first opening 71. The cover 7 is configured to protect the atomizing module 5, while atomized fine droplets discharged from the mist outlet 541 of the atomizing module 5 are dispersed into the external environment through the tubular neck 57.

What the invention claimed is:

1. An ultrasonic atomizer, comprising:
an electrical plug, a control circuit, and an atomizing module;
the atomizing module comprising a piezoelectric vibration module, a mist-guiding tube, a hollow housing, a connecting tube;
the mist-guiding tube comprising a mist outlet at an upper portion thereof;
the hollow housing comprising a tubular neck extending inwardly from an upper portion thereof and an assembling seat at the lower portion thereof;
an upper end of the mist outlet of the mist-guiding tube being movably coupled to the tubular neck in the hollow housing;
the piezoelectric vibration module comprising a piezoelectric vibration element having a plurality of micro-holes formed therein, the piezoelectric vibration module being coupled in the mist-guiding tube;
an upper portion of the piezoelectric vibration module being connected to a lower end of the mist outlet;
an upper portion of the connecting tube being coupled in a lower portion of the mist-guiding tube;
an upper free end of the connecting tube being connected to a lower portion of the piezoelectric vibration module;
the plurality of micro-holes of the piezoelectric vibration element, the mist outlet, the tubular neck, and the connecting tube collectively defining a communicating channel;
a lower portion of the connecting tube being movably coupled to an upper portion of the assembling seat;
the electrical plug being coupled to a side of the hollow housing;
the electrical plug being electrically connected to the control circuit, and the control circuit being electrically connected to the piezoelectric vibration element of the piezoelectric vibration module via a conductive path.

2. The ultrasonic atomizer as claimed in claim 1, wherein the electrical plug is configured to be inserted into an electrical receptacle to provide power to the control circuit and the control circuit is configured to supplying power to the piezoelectric vibration element of the atomizing module via the conductive path.

3. The ultrasonic atomizer as claimed in claim 1, wherein the atomizing module further comprises an elastic member, the elastic member being compressed within the hollow housing and disposed between the upper portion of the hollow housing and the upper portion of the mist-guiding tube to urge the mist-guiding tube downwardly.

4. The ultrasonic atomizer as claimed in claim 3, wherein a gap is predetermined between the lower portion of the connecting tube and the upper portion of the assembling seat, and the elastic force of the elastic member drives the mist-guiding tube and the connecting tube downward, while the lower end of the mist outlet pushes downward against the piezoelectric vibration element of the piezoelectric vibration module so that the piezoelectric vibration element moves downward within a distance of the gap.

5. The ultrasonic atomizer as claimed in claim 3, wherein a protruding rib is formed on an outer side of the mist-guiding tube, the elastic member storing elastic force and having one side thereof sleeved around the outer periphery of a lower portion of the tubular neck, and another side thereof sleeved on the upper portion of the mist-guiding tube and abutting against the protruding rib.

6. The ultrasonic atomizer as claimed in claim 1, wherein the atomizing module is configured to be coupled to a liquid container, wherein the liquid container is detachably coupled to the assembling seat, the liquid container having a bottle opening and a liquid-conducting element, a first end portion of the liquid-conducting element being disposed in liquid within the liquid container, and a second end portion thereof extending outward from the bottle opening and passing through the connecting tube, and a free end of the second end portion of the liquid-conducting element protruding from an upper portion of the connecting tube so as to be connected to the lower portion of the piezoelectric vibration element.

7. The ultrasonic atomizer as claimed in claim 6, wherein the liquid-conducting element comprises any one of a cotton wick, a PET fiber rod, a paper rod, a foam rod, or a porous liquid-conducting element made of a material having a porous structure or capillary action.

8. The ultrasonic atomizer as claimed in claim 6, wherein the piezoelectric vibration module comprises a fixing sleeve, the fixing sleeve being provided with an opening, the piezoelectric vibration element being disposed in the fixing sleeve, the plurality of micro-holes of the piezoelectric vibration element corresponding to the opening of the fixing sleeve, the upper portion of the opening being connected to the lower end of the mist outlet, and the lower portion of the opening being connected to the upper portion of the connecting tube.

9. The ultrasonic atomizer as claimed in claim 8, wherein the diameter of the free end of the second end portion of the liquid-conducting element is smaller than the diameter of the opening of the fixing sleeve, whereby the free end of the second end portion of the liquid-conducting element can be coupled in the opening and contact the piezoelectric vibration element and the plurality of micro-holes thereof in a circumferentially confined manner.

10. The ultrasonic atomizer as claimed in claim 1, wherein the electrical plug is rotatably assembled to the atomizing module so that an angle thereof can be changed, the electrical plug comprises a rotating portion and a USB plug, the rotating portion includes an annular rail formed on an outer peripheral edge, and the rotating portion is formed with a plug hole, the USB plug being coupled in the plug hole; one side of the hollow housing extends with a connecting pipe, an inner side of the connecting pipe is formed with a concave rail, and the annular rail of the rotating portion is rotatably coupled in the concave rail on the inner side of the connecting pipe.

11. The ultrasonic atomizer as claimed in claim 10, wherein the control circuit comprises, an oscillator circuit and a driver circuit.

12. The ultrasonic atomizer as claimed in claim 10, wherein the electrical plug is configured to be detachably coupled to a USB electrical receptacle in different orientations, such that the ultrasonic atomizer can rotate by an angle relative to the USB electrical receptacle from between about 1 degree to 360 degrees, whereby the electrical plug is rotatable to maintain the liquid container in an upright orientation during use.

13. The ultrasonic atomizer as claimed in claim 10, wherein a damping or frictional effect generated when the annular rail and the concave rail relatively rotate is used to movably fix a connection angle between the electrical plug and the hollow housing.

14. The ultrasonic atomizer as claimed in claim 10, wherein a protrusion is formed on a surface of the annular rail, and correspondingly, at least a stopper is formed at 90 degrees or 180 degrees or 270 degrees position of the concave rail, whereby when the rotating portion drives the annular rail and the protrusion thereof to rotate to one side in 90 degrees or 180 degrees or 270 degrees direction, the protrusion is movably engaged with the stopper.

15. The ultrasonic atomizer as claimed in claim 14, wherein two stoppers are formed at 90 degrees or 180 degrees or 270 degrees position of the concave rail, whereby when the rotating portion drives the annular rail and the protrusion thereof to rotate to one side in 90 degrees or 180 degrees or 270 degrees direction, the protrusion is movably engaged with the stoppers.

16. The ultrasonic atomizer as claimed in claim 1, wherein the electrical plug is rotatably assembled to the atomizing module so that an angle thereof can be changed, the electrical plug comprises a rotating portion and an AC plug, the rotating portion includes an annular rail formed on an outer peripheral edge, and the rotating portion is formed with two plug holes, the AC plug being coupled in the plug holes; one side of the hollow housing extends with a connecting pipe, an inner side of the connecting pipe is formed with a concave rail, and the annular rail of the rotating portion is rotatably coupled in the concave rail on the inner side of the connecting pipe.

17. The ultrasonic atomizer as claimed in claim 16, wherein the control circuit comprises, an oscillator circuit, a driver circuit and a step-down circuit.

18. The ultrasonic atomizer as claimed in claim 16, wherein the electrical plug is configured to be detachably coupled to an Ac electrical receptacle in different orientations, such that the Ultrasonic atomizer can rotate by an angle relative to the Ac electrical receptacle from between about 1 degree to 360 degrees, whereby the electrical plug is rotatable to maintain the liquid container in an upright orientation during use.

19. The ultrasonic atomizer as claimed in claim 16, wherein a protrusion is formed on a surface of the annular rail, and correspondingly, at least a stopper is formed at a 90 degrees or 180 degrees or 270 degrees position on either side of the concave rail, whereby when the rotating portion drives the annular rail and the protrusion thereof to rotate to one side in a 90 degrees or 180 degrees or 270 degrees direction, the protrusion is movably engaged with the stopper.

20. The ultrasonic atomizer as claimed in claim 1, wherein the atomizer further comprises a cover, the cover is formed with a first opening on the upper portion thereof, the cover is coupled to the hollow housing, the first opening is sleeved around the out periphery of an upper portion of the tubular neck.
